SEQUENCE LISTING (23 sequences)

SEQ ID NO: 1 - DNA, 1872 bp, Mycobacterium tuberculosis
  32 KD serine protease MTB32A
gactacgttggtgtagaaaaatcctgccgcccggacccttaaggctgggacaatttctgatagctaccccgacacaggaggttacgggatgagcaattcgcgccgccgctcactcaggtggtcatggttgctgagcgtgctggctgccgtcgggctgggcctggccacggcgccggcccaggcggccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgattcgggcgggcccgtcgtcaacggcctaggacaggtggtcggtatgaacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggcctgatttcgtcgcggataccacccgccggccggccaattggattggcgccagccgtgattgccgcgtgagcccccgagttccgtctcccgtgcgcgtggcatcgtggaagcaatgaacgaggcagaacacagcgtcgagcaccctcccgtgcagggcagtcacgtcgaaggcggtgtggtcgagcatccggatgccaaggacttcggcagcgccgccgccctgcccgccgatccgacctggtttaagcacgccgtcttctacgaggtgctggtccgggcgttcttcgacgccagcgcggacggttccggcgatctgcgtggactcatcgatcgcctcgactacctgcagtggcttggcatcgactgcatctggttgccgccgttctacgactcgccgctgcgcgacggcggttacgacattcgcgacttctacaaggtgctgcccgaattcggcaccgtcgacgatttcgtcgccctggtcgacgccgctcaccggcgaggtatccgcatcatcaccgacctggtgatgaatcacacctcggagtcgcacccctggtttcaggagtcccgccgcgacccagacggaccgtacggtgactattacgtgtggagcgacaccagcgagcgctacaccgacgcccggatcatcttcgtcgacaccgaagagtcgaactggtcattcgatcctgtccgccgacagttctactggcaccgattctt

SEQ ID NO: 2 - protein, 355 aa, Mycobacterium tuberculosis
  32 KD serine protease MTB32A
MSNSRRRSLRWSWLLSVLAAVGLGLATAPAQAAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 3 - DNA, 396 bp, Mycobacterium tuberculosis
  14 KD C-terminal fragment of MTB32A Ra12
acggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggcc

SEQ ID NO: 4 - protein, 132 aa, Mycobacterium tuberculosis
  14 KD C-terminal fragment of MTB32A Ra12
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 5 - DNA, 702 bp, Artificial Sequence
  Description of Artificial SequenceRa12-DPPD fusion polypeptide
catatgcatcaccatcaccatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcgacgacgacgacaaggatccacctgacccgcatcagccggacatgacgaaaggctattgcccgggtggccgatggggttttggcgacttggccgtgtgcgacggcgagaagtaccccgacggctcgttttggcaccagtggatgcaaacgtggtttaccggcccacagttttacttcgattgtgtcagcggcggtgagcccctccccggcccgccgccaccgggtggttgcggtggggcaattccgtccgagcagcccaacgctccctgagaattc

SEQ ID NO: 6 - protein, 230 aa, Artificial Sequence
  Description of Artificial SequenceRa12-DPPD fusion polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFDDDDKDPPDPHQPDMTKGYCPGGRWGFGDLAVCDGEKYPDGSFWHQWMQTWFTGPQFYFDCVSGGEPLPGPPPPGGCGGAIPSEQPNAP

SEQ ID NO: 7 - DNA, 1746 bp, Artificial Sequence
  Description of Artificial SequenceRa12-WT1 fusion
catatgcatcaccatcaccatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcccgctggtgccgcgcggcagcccgatgggctccgacgttcgggacctgaacgcactgctgccggcagttccgtccctgggtggtggtggtggttgcgcactgccggttagcggtgcagcacagtgggctccggttctggacttcgcaccgccgggtgcatccgcatacggttccctgggtggtccggcaccgccgccggcaccgccgccgccgccgccgccgccgccgcactccttcatcaaacaggaaccgagctggggtggtgcagaaccgcacgaagaacagtgcctgagcgcattcaccgttcacttctccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttactcaggctgcaataagagatattttaagctgtcccacttacagatgcacagcaggaagcacactggtgagaaaccataccagtgtgacttcaaggactgtgaacgaaggttttttcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttgagaattc

SEQ ID NO: 8 - protein, 578 aa, Artificial Sequence
  Description of Artificial SequenceRa12-WT1 fusion polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYSGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 9 - DNA, 672 bp, Artificial Sequence
  Description of Artificial SequenceRa12-human mammaglobin fusion
catatgcatcaccatcaccatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcatcgagggaaggggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaagaattc

SEQ ID NO: 10 - protein, 220 aa, Artificial Sequence [sequence omitted]

SEQ ID NO: 11 - DNA, 2191 bp, Artificial Sequence
  Description of Artificial SequenceRa12-H9-32A fusion (Ra12-MTB39-MTB32A(N-ter) fusion)
atgcatcaccatcaccatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcagccggcgatatcgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgattcgggcgggcccgtcgtcaacggcctaggacaggtggtcggtatgaacacggccgcgtcctagg

SEQ ID NO: 12 - protein, 729 aa, Artificial Sequence
  Description of Artificial SequenceRa12-H9-32A fusion polypeptide (Ra12-MTB39-MTB32A(N-ter) fusion polypeptide)
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 13 - DNA, 51 bp, Artificial Sequence
  Description of Artificial Sequence oligonucleotide primer for PCR amplification of Ra12 C-terminal fragment of MTB32A
caattacatatgcatcaccatcaccatcacacggccgcgtccgataacttc

SEQ ID NO: 14 - DNA, 33 bp, Artificial Sequence
  Description of Artificial Sequence3′ oligonucleotide primer for PCR amplification of Ra12 C-terminal fragment of MTB32A
ctaatcgaattcggccgggggtccctcggccaa

SEQ ID NO: 15 - DNA, 48 bp, Artificial Sequence
  Description of Artificial Sequence5′ oligonucleotide primer containing enterokinase recognition site for PCR amplification of DPPD mature secreted form
caattagaattcgacgacgacgacaaggatccacctgacccgcatcag

SEQ ID NO: 16 - DNA, 33 bp, Artificial Sequence
  Description of Artificial Sequence3′ oligonucleotide primer containing enterokinase recognition site for PCR amplification of DPPD mature secreted form
caattagaattctcagggagcgttgggctgctc

SEQ ID NO: 17 - protein, 30 aa, Artificial Sequence
  Description of Artificial SequenceRa12(short) polypeptide
TAASDNFQLSQGGQGFAIPIGQAMAIAGQI

SEQ ID NO: 18 - protein, 128 aa, Artificial Sequence
  Description of Artificial SequenceRa12(long) polypeptide
TAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 19 - DNA, 34 bp, Artificial Sequence
  Description of Artificial Sequence5′ oligonucleotide primer, HindIII site, for PCR amplification of human mammaglobin
gcgaagcttatgaagttgctgatggtcctcatgc

SEQ ID NO: 20 - DNA, 36 bp, Artificial Sequence
  Description of Artificial Sequence3′ oligonucleotide primer, XhoI site, for PCR amplification of human mammaglobin
cggctcgagttaaaataaatcacaaagactgctgtc

SEQ ID NO: 21 - protein, 7 aa, Artificial Sequence
  Description of Artificial SequenceMet-His tag 6aa
MHHHHHH

SEQ ID NO: 22 - protein, 4 aa, Artificial Sequence
  Description of Artificial Sequenceenterokinase recognition site
DDDK

SEQ ID NO: 23 - protein, 128 aa, Mycobacterium tuberculosis
  positions 1-128 of Ra12
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAE

(19) United States
(12) Patent Application Publication
(10) Pub. No.: US 2006/0040356 A1
(43) Pub. Date: Feb. 23, 2006

(54) Methods of using a Mycobacterium tuberculosis coding sequence to facilitate stable and high yield expression of the heterologous proteins

(75) Inventors: Yasir Skeiky, Seattle, WA (US); Jeffrey Guderian, Lynnwood, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corporation, Seattle, WA (US)

(21) Appl. No.: 11/222,451

(22) Filed: Sep. 7, 2005

Related U.S. Application Data
(62) Division of application No. 09/684,215, filed on Oct. 6, 2000.
(60) Provisional application No. 60/158,585, filed on Oct. 7, 1999.

(51) Int. Cl. C12P 21/06; C07H 21/04; C12N 15/74; C12N 1/21; C07K 14/35

(52) U.S. Cl. 435/69.1; 435/320.1; 435/252.3; 530/350; 536/23.7

(57) ABSTRACT

The present invention relates generally to nucleic acid and amino acid sequences of a fusion polypeptide comprising a Mycobacterium tuberculosis polypeptide, and a heterologous polypeptide of interest, expression vectors and host cells comprising such nucleic acids, and methods for producing such fusion polypeptides. In particular, the invention relates to materials and methods of using such M. tuberculosis sequence as a fusion partner to facilitate the stable and high yield expression of recombinant heterologous polypeptides of both eukaryotic and prokaryotic origin.

### CROSS-REFERENCES TO RELATED APPLICATIONS

This application claims priority to provisional application U.S. Ser. No. 60/158,585, filed Oct. 7, 1999, the disclosure of which is herein incorporated by reference in its entirety.

### TECHNICAL FIELD

The present invention relates generally to nucleic acid and amino acid sequences of a fusion polypeptide comprising a Mycobacterium tuberculosis polypeptide, and a heterologous polypeptide of interest, expression vectors and host cells comprising such nucleic acids, and methods for producing such fusion polypeptides. In particular, the invention relates to materials and methods of using such M. tuberculosis sequence as a fusion partner to facilitate the stable and high yield expression of recombinant heterologous polypeptides of both eukaryotic and prokaryotic origin.

### BACKGROUND OF THE INVENTION

The advent of recombinant DNA technology has led to the molecular cloning of a large number of coding sequences or genes from diverse cell types. In order to study the function of these genes or to produce the products encoded by such sequences, these genes are inserted in expression vectors under the control of appropriate regulatory sequences. This transfer of the expression vector into a eukaryotic or prokaryotic host cell generally results in the expression of the encoded product which can be subsequently purified. Large-scale production of many gene products is particularly important in cases where such products are of medical or industrial value.

However, notwithstanding the advances in gene expression, certain coding sequences do not readily produce their products in stable form. For example, expression in E. coli of recombinant proteins could be problematic particularly for proteins with trans-membrane domains or extensive hydrophobic sequences. Moreover, recombinant proteins may not contain the N-terminal amino acid residues with the appropriate codon bias. Thus, there remains a need for improved materials and methods for the expression of recombinant proteins.

### SUMMARY OF THE INVENTION

The present invention provides for the first time recombinant nucleic acid molecules that encode fusion polypeptides comprising a Ra12 polypeptide and a heterologous polypeptide, fusion polypeptides, expression vectors and host cells comprising the nucleic acid molecules. The present invention further provides methods of using such recombinant nucleic acid molecules, expression vectors, and host cells to produce stable and high yield expression of fusion polypeptides of interest.

In one aspect, the present invention provides recombinant nucleic acid molecules that encode a fusion polypeptide, the recombinant nucleic acid molecules comprising a Ra12 polynucleotide sequence and a heterologous polynucleotide sequence, wherein the Ra12 polynucleotide sequence hybridizes to SEQ ID NO:3 under stringent conditions. In one embodiment, the recombinant nucleic acid molecules comprise a Ra12 polynucleotide sequence which is located 5′ to a heterologous polynucleotide sequence. In another embodiment, the recombinant nucleic acid molecules further comprise a polynucleotide sequence that encodes a linker peptide between the Ra12 polynucleotide sequence and the heterologous polynucleotide sequence, wherein the linker peptide may comprise a cleavage site. In yet another embodiment, the recombinant nucleic acid molecules encode fusion polypeptides which further comprise an affinity tag. In yet another embodiment, the recombinant nucleic acid molecules encode a fusion polypeptide comprising a DPPD, a WT1, a mammaglobin, or a H9-32A heterologous polypeptide. In yet another embodiment, the recombinant nucleic acid molecules comprise a Ra12 polynucleotide sequence comprising at least about 30 nucleotides, at least about 60 nucleotides, or at least about 100 nucleotides. In yet another embodiment, the recombinant nucleic acid molecules comprise a Ra12 polynucleotide sequence as shown in SEQ ID NO:3. In yet another embodiment, the recombinant nucleic acid molecules comprise a Ra12 polynucleotide sequence that encodes a Ra12 polynucleotide as shown in SEQ ID NO:4, SEQ ID NO:17 or SEQ ID NO:18.

In another aspect, the present invention provides expression vectors comprising a promoter operably linked to a recombinant nucleic acid molecule according to any one of embodiments described herein.

In yet another aspect, the present invention provides host cells comprising expression vectors according to any one of embodiments described herein. In a preferred embodiment, the host cell is E. coli.

In yet another aspect, the present invention provides fusion polypeptides comprising a Ra12 polypeptide and a heterologous polypeptide, wherein the Ra12 polypeptide is encoded by a Ra12 polynucleotide sequence that hybridizes to SEQ ID NO:3 under stringent hybridization conditions. In one embodiment, the Ra12 polypeptide comprises at least about 10 amino acids, at least about 30 amino acids, or at least about 100 amino acids. In another embodiment, the Ra12 polypeptide has a sequence as shown in SEQ ID NO:4, SEQ ID NO:17, or SEQ ID NO:18.

In yet another aspect, the present invention provides methods of producing fusion polypeptides, the method comprising expressing in a host cell a recombinant nucleic acid molecule that encodes a fusion polypeptide, the fusion polypeptide comprising a Ra12 polypeptide and a heterologous polypeptide, wherein the Ra12 polypeptide is encoded by a Ra12 polynucleotide sequence that hybridizes to SEQ ID NO:3 under stringent conditions. In one embodiment, the method further comprises purifying fusion polypeptides after their expression. In another embodiment, the method further comprises cleaving a fusion polypeptide between a Ra12 polypeptide and a heterologous polypeptide.

These and other aspects of the present invention will become apparent upon reference to the following detailed description and attached drawings.

### BRIEF DESCRIPTION OF THE DRAWINGS

FIG. 1 illustrates a nucleotide sequence (SEQ ID NO:1) and an amino acid sequence (SEQ ID NO:2) of MTB32A.

FIG. 2 illustrates a nucleotide sequence (SEQ ID NO:3) and an amino acid sequence (SEQ ID NO:4) of Ra12.

FIG. 3 illustrates a recombinant nucleic acid sequence comprising a nucleotide sequence (SEQ ID NO:5) and an amino acid sequence (SEQ ID NO:6) of Ra12-DPPD fusion polypeptide.

FIG. 4 illustrates a recombinant nucleic acid sequence comprising a nucleotide sequence (SEQ ID NO:7) and an amino acid sequence (SEQ ID NO:8) of Ra12-WT1 fusion polypeptide.

FIG. 5 illustrates a recombinant nucleic acid sequence comprising a nucleotide sequence (SEQ ID NO:9) and an amino acid sequence (SEQ ID NO:10) of Ra12-mammaglobin fusion polypeptide.

FIG. 6 illustrates a recombinant nucleic acid sequence comprising a nucleotide sequence (SEQ ID NO:11) and an amino acid sequence (SEQ ID NO:12) of Ra12-H9-32A fusion polypeptide.

FIG. 7 illustrates Ra12(short) polypeptide (SEQ ID NO:17), which has amino acids 1-30 of SEQ ID NO:3.

FIG. 8 illustrates Ra12(long) polypeptide (SEQ ID NO:18), which has amino acids 1-128 of SEQ ID NO:4.

FIG. 9 illustrates a construct of Ra12 (short) polynucleotide fused to a human mammaglobin gene.

### DETAILED DESCRIPTION OF THE INVENTION

As noted above, the present invention provides for the first time recombinant nucleic acid molecules, expression vectors, host cells, fusion polypeptides, and methods for producing fusion polypeptides, using a Mycobacterium tuberculosis coding sequence, namely a Ra12 nucleic acid which is a subsequence of a MTB32A nucleic acid. In particular, the invention provides materials and methods for using Ra12 sequences as a fusion partner to facilitate the stable and high yield expression of recombinant heterologous polypeptides of both eukaryotic and prokaryotic origin.

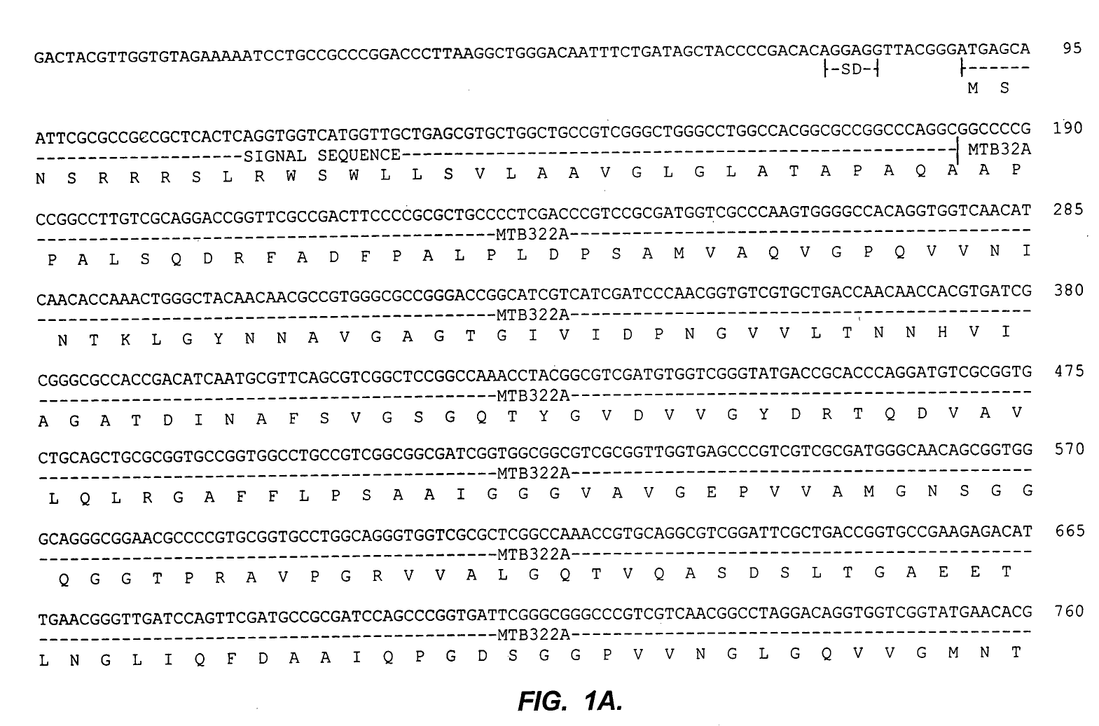

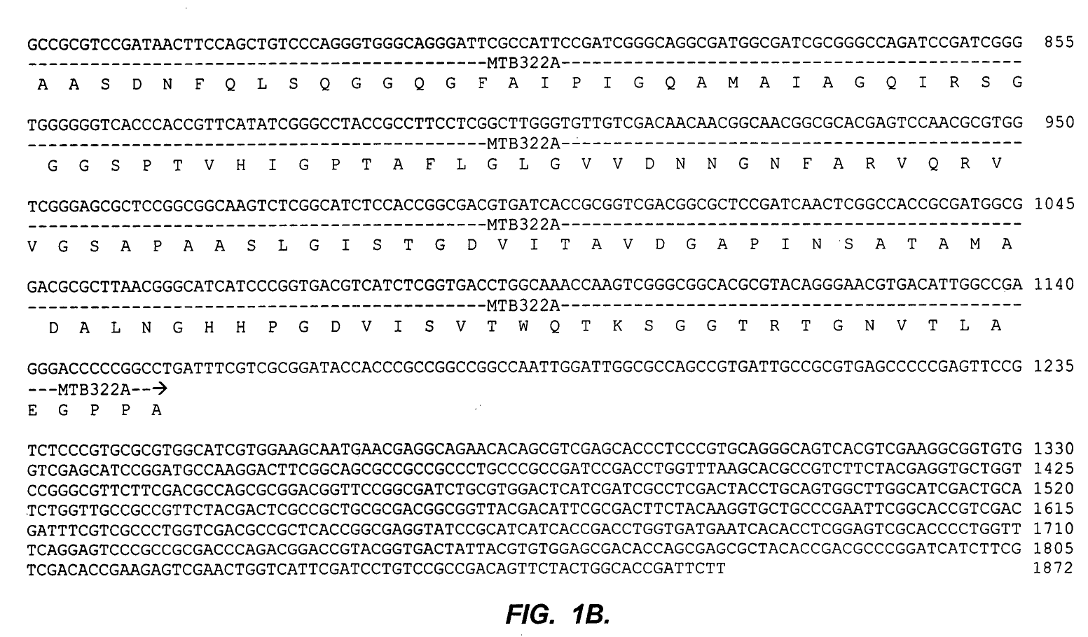

MTB32A is a serine protease of 32 KD molecular weight encoded by a gene in virulent and avirulent strains of M. tuberculosis. The complete nucleotide sequence (SEQ ID NO:1) and amino acid sequence (SEQ ID NO:2) of MTB32A are disclosed in FIG. 1. See, also, Skeiky et al., Infection and Immun. (1999) 67:3998-4007, incorporated herein by reference. This protein is naturally secreted into the supernatant of bacterial cultures. The open reading frame of the coding sequence contains N-terminal hydrophobic secretory signals. It stimulates peripheral blood mononuclear cells from healthy purified protein derivative (PPD)-positive donors to proliferate and secrete interferon. Thus, MTB32A is a candidate antigen for use in vaccine development against tuberculosis.

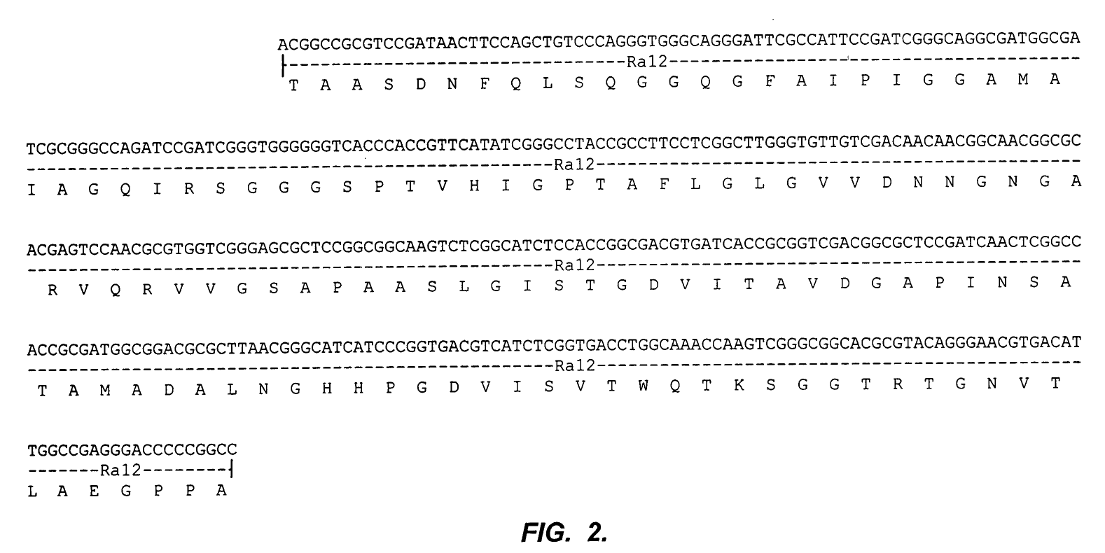

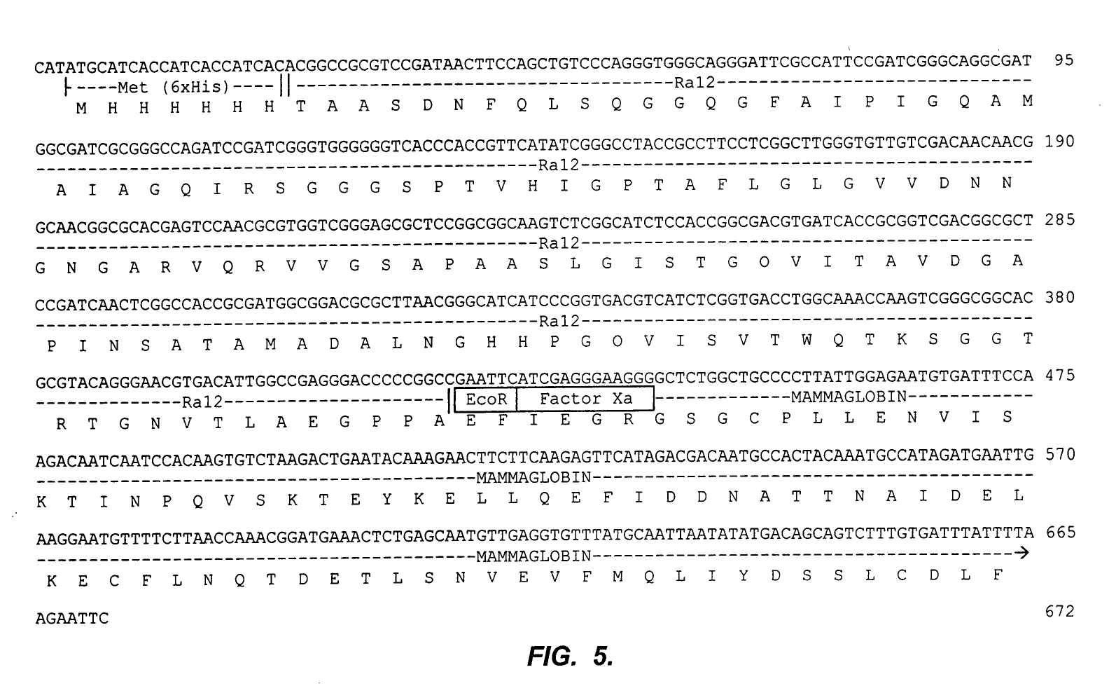

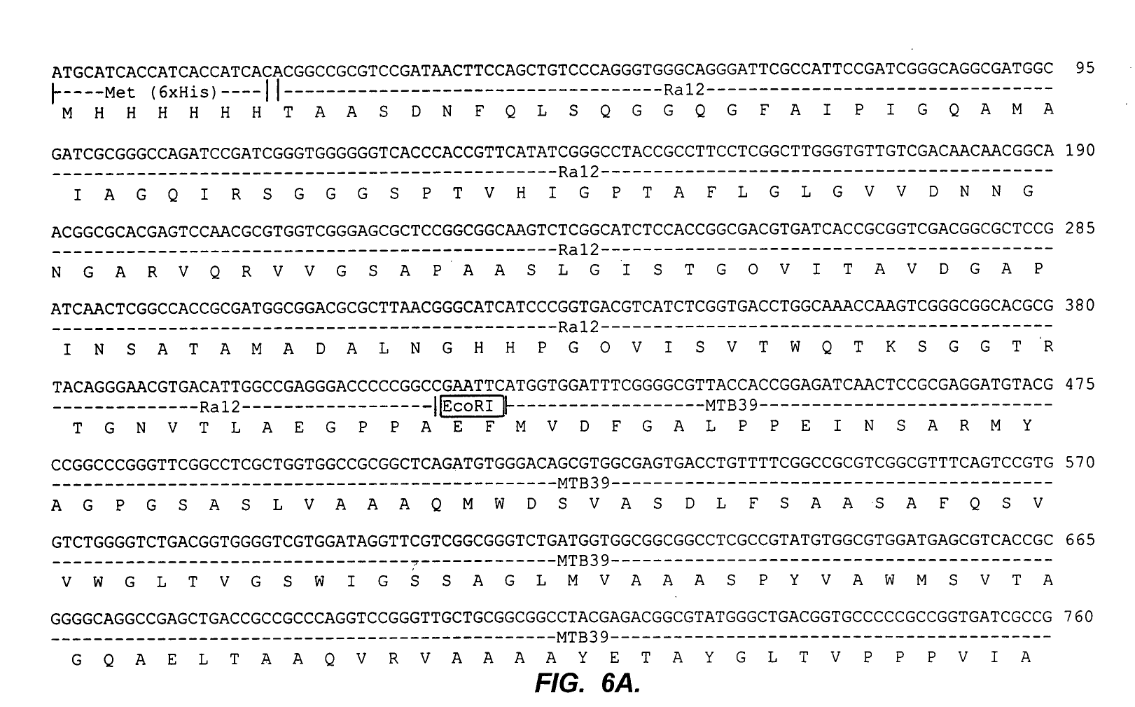

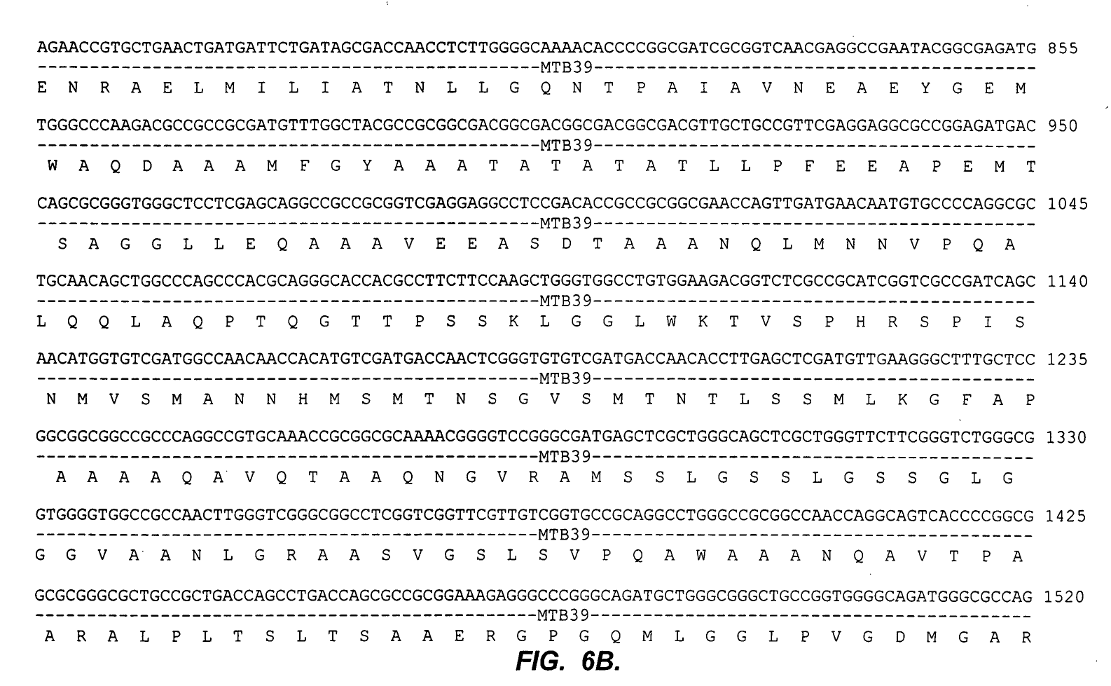

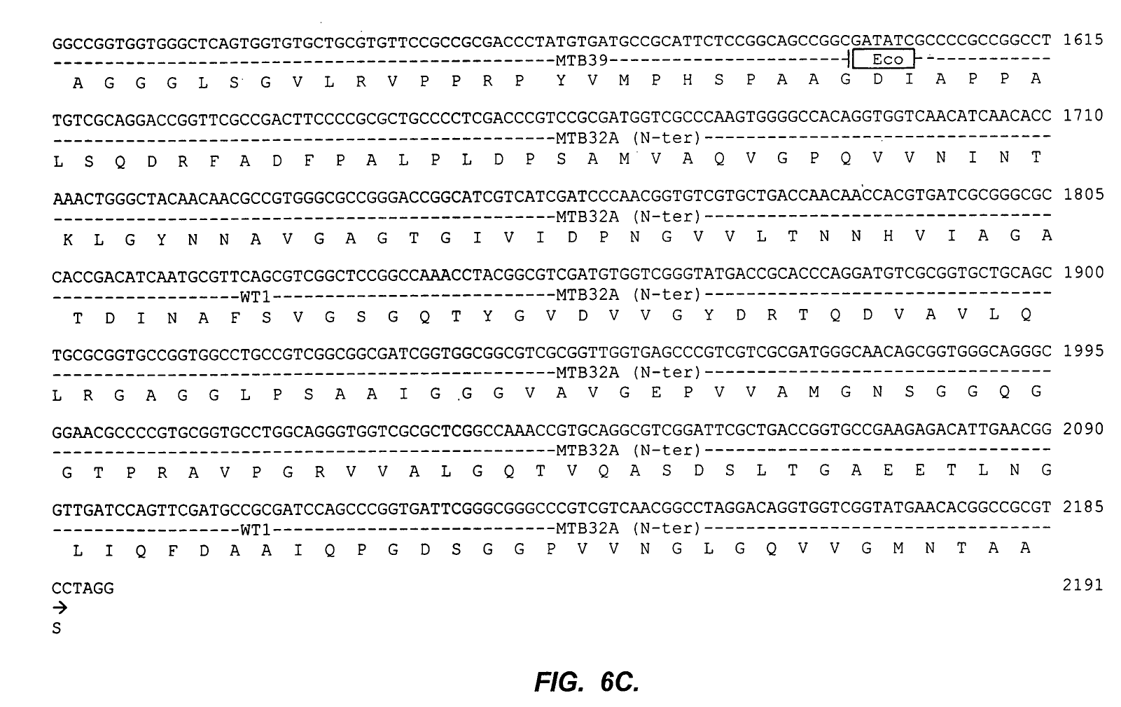

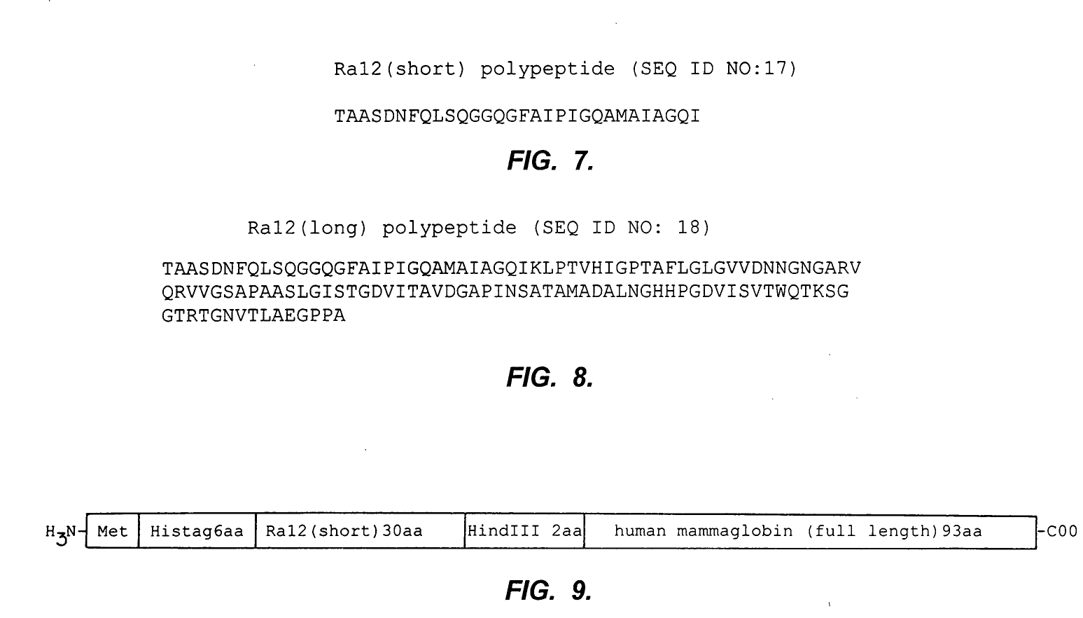

Surprisingly, it was discovered by the present inventors that a 14 KD C-terminal fragment of the MTB32A coding sequence expresses at high levels on its own and remains as a soluble protein throughout the purification process. This 14 KD C-terminal fragment of the MTB32A is referred herein as Ra12 (having amino acid residues 192 to 323 of MTB32A). The nucleic acid and amino acid sequences of native Ra12 are shown, e.g., in FIGS. 2-6. As described in detail below, the term “Ra12 polypeptide” or “Ra12 polynucleotide” as used herein refer to the native Ra12 sequences (e.g., SEQ ID NO:3 or SEQ ID NO:4), their variants, or fragments thereof (e.g., SEQ ID NO:17 or SEQ ID NO:18). The present invention utilizes these properties of Ra12 polypeptides and provides recombinant nucleic acid molecules, expression vectors, host cells, and methods for stable and high yield expression of fusion polypeptides comprising a Ra12 polypeptide and a heterologous polypeptide of interest. The materials and methods of the present invention are particularly useful in expressing certain heterologous polypeptides (e.g., DPPD) that other conventional expression methods failed to express in any substantial quantity.

Recombinant Fusion Nucleic Acids

Recombinant nucleic acids, which encode a fusion polypeptide comprising a Ra12 polypeptide and a heterologous polypeptide of interest, can be readily constructed by conventional genetic engineering techniques. Recombinant nucleic acids are constructed so that, preferably, a Ra12 polynucleotide sequence is located 5′ to a selected heterologous polynucleotide sequence. It may also be appropriate to place a Ra12 polynucleotide sequence 3′ to a selected heterologous polynucleotide sequence or to insert a heterologous polynucleotide sequence into a site within a Ra12 polynucleotide sequence.

In the present invention, any suitable heterologous polynucleotide of interest can be selected as a fusion partner to Ra12 nucleic acids to produce a fusion polypeptide. A “heterologous sequence” or a “heterologous nucleic acid,” as used herein, is one that originates from a source foreign to the particular host cell, or, if from the same source, is modified from its original form. Thus, a heterologous nucleic acid in a prokaryotic host cell includes a heterologous nucleic acid that is endogenous to particular host cell that has been modified. Modification of the heterologous sequence may occur, e.g., by treating the DNA with a restriction enzyme to generate a DNA fragment that is capable of being operably linked to the promoter. Techniques such as site-directed mutagenesis are also useful in modifying a heterologous sequence.

A heterologous nucleic acid from both eukaryotic and prokaryotic origins can be selected as a fusion partner. These nucleic acids include, but are not limited to, nucleic acids that encode pathogenic antigens, bacterial antigens, viral antigens, cancer antigens, tumor antigens, and tumor suppressors. Exemplary heterologous nucleic acids of interest include DPPD, WT1, mammaglobin, H9-32A nucleic acids, and other Mycobacterium tuberculosis nucleic acids (see, e.g., Cole et al. Nature (1999) 393:537-544; http://www.sanger.ac.uk; and http://www.pasteur.fr/mycdb/ for the complete genome sequences of M. tuberculosis; see, also WO98/53075 and WO98/53076, both of which are published on Nov. 26, 1998 for nucleic acid sequences that encode M. tuberculosis proteins). Any one of the nucleic acids disclosed herein can be used alone or in combination as a heterologous nucleic acid that can be selected as a fusion partner.

In addition, any suitable Ra12 polynucleotide (e.g., native Ra12 polynucleotide having SEQ ID NO:3, variants or fragments thereof) can be used in constructing recombinant fusion nucleic acids of the present invention. Preferred Ra12 polynucleotides comprise at least about 15 consecutive nucleotides, at least about 30 nucleotides, at least about 60 nucleotides, at least about 100 nucleotides, at least about 200 nucleotides, or at least about 300 nucleotides. Polynucleotides may be single-stranded or double-stranded, and may be DNA (genomic, cDNA or synthetic) or RNA molecules.

In one embodiment, the Ra12 polynucleotide sequence is as shown in SEQ ID NO:3. In another embodiment, the Ra12 polynucleotide sequence encodes a Ra12 polypeptide as shown in SEQ ID NO:4. In some embodiments, the Ra12 polynucleotide sequence comprises a portion of SEQ ID NO:3 or encodes a portion of SEQ ID NO:4. For instance, a Ra12 polynucleotide comprising 90 nucleotides (e.g., nucleotides 1-90 of SEQ ID NO:3), or a Ra12 polynucleotide comprising 384 nucleotides (e.g., nucleotides 1-384 of SEQ ID NO:3) can be used as a fusion partner. See Examples 2 and 3 below.

Polynucleotides may comprise a native sequence (i.e., an endogenous sequence that encodes a Ra12 polypeptide SEQ ID NO:3 or a portion thereof) or may comprise a variant of such a sequence. Polynucleotide variants may contain one or more substitutions, additions, deletions and/or insertions such that the biological activity of the encoded fusion polypeptide is not diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide (SEQ ID NO:4) or a portion thereof. Optionally, the identity exists over a region that is at least about 25 to about 50 amino acids or nucleotides in length, or optionally over a region that is 75-100 amino acids or nucleotides in length.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

Preferred examples of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which do not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Variants may also, or alternatively, be substantially homologous to a native Ra12 polynucleotide (e.g., SEQ ID NO:3), or a portion or complement thereof. Such polynucleotide variants are capable of hybridizing under stringent conditions to a naturally occurring DNA sequence encoding a native Ra12 polynucleotide (or a complementary sequence).

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5× SSC, and 1% SDS, incubating at 42° C., or, 5× SSC, 1% SDS, incubating at 65° C., with a wash in 0.2× SSC, and 0.1% SDS at 65° C.

It will be appreciated by those of ordinary skill in the art that, as a result of the degeneracy of the genetic code, there are many nucleotide sequences that encode a Ra12 polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Thus, the terms such as “Ra12 polynucleotide” or “Ra12 polynucleotide sequence” as used herein refer to native Ra12 polynucleotide sequences (e.g., SEQ ID NO:3), fragments thereof, or any variants thereof. Functionally, any Ra12 polynucleotide has the ability to produce a fusion protein, and its ability to produce a fusion proteins in host cells may be enhanced or unchanged, relative to the native Ra12 polynucleotide (e.g., SEQ ID NO:3), or may be diminished by less than 50%, and preferably less than 20%, relative to the native Ra12 polynucleotide.

Nucleic acids encoding Ra12 polypeptides of this invention can be prepared by any suitable method known in the art. Exemplary methods include cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

In one embodiment, a nucleic acid encoding MTB32A or Ra12 is isolated by routine cloning methods. Nucleotide sequences of MTB32A or Ra12 as provided herein are used to provide probes that specifically hybridize to other MTB32A or Ra12 nucleic acids in a genomic DNA sample, or to a MTB32A mRNA or Ra12 mRNA in a total RNA sample (e.g., in a Southern or Northern blot). Once the target MTB32A or Ra12 nucleic acids are identified, it can be isolated according to standard methods known to those of skill in the art.

The desired nucleic acids can also be cloned using well known amplification techniques. Examples of protocols sufficient to direct persons of skill through in vitro amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039. Suitable primers for use in the amplification of the nucleic acids of the invention can be designed based on the sequences provided herein.

The MTB32A or Ra12 nucleic acids can also be cloned by detecting their expressed product by means of assays based on the physical, chemical, or immunological properties of the expressed protein. For example, one can identify a cloned MTB32A or Ra12 nucleic acid by the ability of a polypeptide encoded by the nucleic acid to bind with antisera or purified antibodies made against the MTB32A or Ra12 polypeptides provided herein, which also recognize and selectively bind to the MTB32A or Ra12 homologs.

In some embodiments, it may be desirable to modify the MTB32A or Ra12 nucleic acids of the invention. Altered nucleotide sequences which can be used in accordance with the invention include deletions, additions or substitutions of different nucleotide residues resulting in a sequence that encodes the same or a functionally equivalent gene product. The gene product itself may contain deletions, additions or substitutions of amino acid residues, which result in a silent change thus producing a functionally equivalent antigenic epitope. Such conservative amino acid substitutions may be made on the basis of similarity in polarity, charge, solubility, hydrophobicity, hydrophilicity, and/or the amphipathic nature of the residues involved. Preferably, Ra12 nucleic acids that are shorter in length than SEQ ID NO:3 that encode biologically active fusion partner can be used. Such smaller functional equivalents of Ra12 polypeptides may be desirable to increase the amount of host cell resources that are available for the production of heterologous polypeptides of interest.

One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith (1979) Gene 8:81-97, Roberts et al. (1987) Nature 328: 731-734.

Recombinant nucleic acids that encode a fusion polypeptide comprising a Ra12 polypeptide and a selected heterologous polypeptide can be prepared using any methods known in the art. As described above, recombinant nucleic acids are constructed so that a Ra12 polynucleotide sequence is located in any suitable place in a construct. Preferably, a Ra12 polynucleotide sequence is located 5′ to a selected heterologous polynucleotide sequence. Ra12 and heterologous polynucleotide sequences can also be modified to facilitate their fusion and subsequent expression of fusion polypeptides. For example, the 3′ stop codon of the Ra12 polynucleotide sequence can be substituted with an in frame linker sequence, which may provide restriction sites and/or cleavage sites. The recombinant nucleic acids can further comprise other nucleotide sequences such as sequences that encode affinity tags to facilitate protein purification protocol.

Expression Vectors and Host Cells

The recombinant nucleic acids as described herein can be joined to a variety of other nucleotide sequences using established recombinant DNA techniques. For example, a polynucleotide can be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend on the desired use, and will be apparent to those of ordinary skill in the art.

DNA sequences encoding the polypeptide components may be assembled separately, and ligated into an appropriate expression vector. The 3′ end of the DNA sequence encoding one polypeptide component is ligated, with or without a polynucleotide sequence encoding a peptide linker, to the 5′ end of a DNA sequence encoding the second polypeptide component so that the reading frames of the sequences are in phase. This permits translation into a single fusion protein that retains the biological activity of both component polypeptides.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter) and the like may be used; when cloning in yeast cell systems, promoters such as ADHI, PGK, PHO5, or the α factor promoter may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of a the antigen coding sequence, SV40-, BPV- and EBV-based vectors may be used with an appropriate selectable marker.

A variety of host-expression vector systems may be utilized to express a Ra12 fusion protein coding sequences. These include, but are not limited to, microorganisms such as bacteria (e.g., E. coli, B. subtilis) transformed with recombinant bacteriophage DNA, plasmid DNA or cosmid DNA expression vectors containing a coding sequence; yeast (e.g., Saccharomycdes, Pichia) transformed with recombinant yeast expression vectors containing a coding sequence; insect cell systems infected with recombinant virus expression vectors (e.g., baculovirus) containing a coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a coding sequence; or mammalian cell systems (e.g., COS, CHO, BHK, 293, 3T3 cells). The expression elements of these systems vary in their strength and specificities.

Bacterial systems are preferred for the expression of Ra12 fusion polypeptides. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer et al., Proc. Natl. Acad. Sci. U.S.A. (1983) 80:21-25); and the lambda-derived P_{L} promoter and N-gene ribosome binding site (Shimatake et al., Nature (1981) 292: 128). The particular promoter system is not critical to the invention, any available promoter that functions in prokaryotes can be used.

Either constitutive or regulated promoters can be used in the present invention. Regulated promoters can be advantageous because the host cells can be grown to high densities before expression of the Ra12 fusion polypeptides is induced. High level expression of heterologous proteins slows cell growth in some situations. Regulated promoters especially suitable for use in E. coli include the bacteriophage lambda P_{L} promoter, the hybrid trp-lac promoter (Amann et al., Gene (1983) 25: 167; de Boer et al., Proc. Natl. Acad. Sci. USA (1983) 80: 21, and the bacteriophage T7 promoter (Studier et al., J. Mol. Biol. (1986); Tabor et al., (1985). These promoters and their use are discussed in Sambrook et al., (1989) Molecular Cloning: A Laboratory Manual, 2nd Ed., Vols. 1-3, Cold Spring Harbor Laboratory.

For expression of Ra12 fusion polypeptides in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

A ribosome binding site (RBS) is conveniently included in the expression cassettes of the invention. An RBS in E. coli, for example, consists of a nucleotide sequence 3-9 nucleotides in length located 3-11 nucleotides upstream of the initiation codon (Shine and Dalgarno, Nature (1975) 254: 34; Steitz, In Biological regulation and development: Gene expression (ed. R. F. Goldberger), vol. 1, p. 349, 1979, Plenum Publishing, N.Y.).

When large quantities of the Ra12 fusion protein are to be produced, vectors which direct the expression of high levels of fusion protein products that are readily purified may be desirable. Such vectors include, but are not limited to, the E. coli expression vector pUR278 (Ruther et al. (1983) EMBO J. 2:1791), in which a coding sequence may be ligated into the vector in frame with the lacZ coding region so that a hybrid protein is produced; pIN vectors (Inouye and Inouye (1985) Nucleic Acids Res. 13:3101-3109; Van Heeke and Schuster (1989) J. Biol. Chem. 264:5503-5509); and the like. pGEX vectors may also be used to express foreign polypeptides as fusion proteins with glutathione S-transferase (GST). In general, such fusion proteins are soluble and can be purified easily from lysed cells by adsorption to glutathione-agarose beads followed by elution in the presence of free glutathione. For certain applications, it may be desirable to cleave the heterologous polypeptide of interest from the Ra12 fusion polypeptide after purification. This can be accomplished by any of several methods known in the art. For example, the pGEX vectors are designed to include thrombin or factor Xa protease cleavage sites so that the cloned fusion polypeptide of interest can be released from the GST moiety. See, e.g., Sambrook et al., supra.; Itakura et al., Science (1977) 198:1056; Goeddel et al., Proc. Natl. Acad. Sci. USA (1979) 76:106; Nagai et al., Nature (1984) 309:810; Sung et al., Proc. Natl. Acad. Sci. USA (1986) 83:561. Cleavage sites can be engineered into the recombinant nucleic acids for the fusion proteins at the desired point of cleavage.

Fusion Polypeptides

Within the context of the present invention, a “fusion” polypeptide comprises at least two parts: a Ra12 polypeptide as described herein, and a heterologous polypeptide of interest. In a fusion polypeptide, a Ra12 polypeptide is preferably fused, directly or indirectly, to the amino terminus of a heterologous polypeptide of interest, although fusion to the carboxy terminus of the heterologous polypeptide or insertion of the heterologous polypeptide into a site within an Ra12 polypeptide may also be appropriate.

Any heterologous polypeptide of interest, either eukaryotic or prokaryotic origins, can be selected as a fusion partner to a Ra12 polypeptide. These heterologous polypeptides include, but are not limited to, pathogenic antigens, bacterial antigens, viral antigens, cancer antigens, tumor antigens, and tumor suppressors. Exemplary heterologous polypeptides include DPPD, WT1, mammaglobin, H9-32A polypeptides, or other M. tuberculosis proteins. Any one of these polypeptides can be used alone or in combination as a heterologous polypeptide that can be selected as a fusion partner.

As noted above, a fusion polypeptide may comprise a native Ra12 polypeptide (e.g., SEQ ID NO:4), a variant thereof, or a fragment thereof. A polypeptide “variant,” as used herein, is a polypeptide that differs from a native Ra12 polypeptide in one or more substitutions, deletions, additions and/or insertions, such that the biological activity of the polypeptide is not substantially diminished. In other words, the ability of a variant to produce fusion polypeptide in host cells may be enhanced or unchanged, relative to the native Ra12 protein, or may be diminished by less than 50%, and preferably less than 20%, relative to the native Ra12 protein. Such variants may generally be identified by modifying one of the above polypeptide sequences and evaluating the level of fusion polypeptide production in host cells, such as in E. coli. Exemplary variants include those in which a small portion (e.g., 1-30 amino acids, preferably 5-15 amino acids) has been removed from the N- and/or C-terminal of the native Ra12 polypeptides. In one embodiment, variants of native Ra12 polypeptides comprise at least about 5 amino acids, at least about 10 amino acids, at least about 30 amino acids, at least about 50 amino acids, or at least about 100 amino acids.

In one embodiment, the Ra12 polypeptide sequence is as shown in SEQ ID NO:4. In another embodiments, the Ra12 polypeptide sequence comprises a portion of SEQ ID NO:4. For instance, an Ra12 polypeptide comprising 30 amino acids (e.g., amino acids 1-30 of SEQ ID NO:4) or an Ra12 polypeptide comprising 128 amino acids (e.g., amino acids 1-128 of SEQ ID NO:4) can be used as a fusion partner. See Examples 2 and 3 below.

Polypeptide variants preferably exhibit at least about 70%, more preferably at least about 80% or at least about 90%, and most preferably at least about 95% identity (determined as described above) to the identified polypeptides. Optionally, identity exists over a region that is at least about 20 to about 50 amino acids in length, or optionally over a region that is 75-100 amino acids in length.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

Thus, the terms such as “Ra12 polypeptide” or “Ra12 polypeptide sequence” as used herein refer to native Ra12 polynucleotide sequences (e.g., SEQ ID NO:4), fragments thereof (e.g., SEQ ID NO:17 or 18), or any variants thereof. Functionally, a Ra12 polypeptide has the ability to produce a fusion protein, and its ability to produce a fusion proteins in host cells may be enhanced or unchanged, relative to the native Ra12 polypeptide (e.g., SEQ ID NO:4), or may be diminished by less than 50%, and preferably less than 20%, relative to the native Ra12 polypeptide.

As noted above, fusion polypeptides may be conjugated to a linker or other sequence for ease of synthesis, purification or identification of the polypeptide or to enhance binding of the polypeptide to a solid support. For example, a peptide linker sequence may be employed to separate a Ra12 polypeptide and a heterologous polypeptide of interest by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. In certain embodiments, peptide linker sequences may contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

In a preferred embodiment, a linker can provide a specific cleavage site between a Ra12 polypeptide and a heterologous polypeptide of interest. Such a cleavage site may contain a target for proteolytic enzyme that includes, for example, enterokinase, Factor Xa, trypsin, collagenase, thrombin, ubiquitin hydrolase; or for chemical cleavage agents such as, for example, cyanogen bromide or hydroxyamine.

A fusion polypeptide may optionally contain an affinity tag which is linked to the fusion polypeptide so that the purification of recombinant polypeptides can be simplified. For example, multiple histidine residues encoded by the tag allow the use of metal chelate affinity chromatography methods for the purification of fusion polypeptides. Other examples of affinity tag molecules include, Strep-tag, PinPoint, maltose binding protein, glutathione S-transferase, etc. See, e.g., Glick and Pasternak (1999) Molecular Biotechnology Principles and Applications of Recombinant DNA, 2^{nd} Ed., American Society for Microbiology, Washington, D.C.

Fusion polypeptides may be prepared using any of a variety of well known techniques. Recombinant fusion polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells described above. Preferably, the host cell employed is E. coli. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

In addition to providing stable and high yield expression of fusion polypeptides of interest, the recombinant fusion nucleic acids and fusion polypeptides of the invention can be used in a number of other methods. For example, the fusion polypeptide coding sequence of the invention can be used to encode a protein product for use as an antigen for detecting serum antibodies. For example, the presence of serum antibodies to M. tuberculosis antigens in an individual indicates that the individual is infected with M. tuberculosis. In standard diagnostic tests, serum antibodies to M. tuberculosis are detected by monitoring binding of serum antibodies to M. tuberculosis proteins. The fusion polypeptides of the invention are useful as sources of proteins for monitoring binding of serum antibodies to fusion proteins.

Alternatively, the fusion polypeptide can be used as an immunogen to induce and/or enhance immune responses. Such coding sequences can be ligated with a coding sequence of another molecule such as a M. tuberculosis antigen, a cytokine or an adjuvant. Such polynucleotides may be used in vivo as a DNA vaccine (U.S. Pat. Nos. 5,589,466; 5,679,647; and 5,703,055). Alternatively, purified or partially purified fusion polypeptides or fragments may be used as vaccines or therapeutic compositions. Any of a variety of methods known in the art can be employed to produce vaccines or therapeutic compositions comprising the fusion polypeptides of the present invention.

Protein Purification and Preparations

Once a recombinant protein is expressed, it can be identified by assays based on the physical or functional properties of the product, including radioactive labeling of the product followed by analysis by gel electrophoresis, radioimmunoassay, ELISA, bioassays, etc.

Once the encoded protein is identified, it may be isolated and purified by standard methods including chromatography (e.g., high performance liquid chromatography, ion exchange, affinity, and sizing column chromatography), centrifugation, differential solubility, or by any other standard technique for the purification of proteins. See, generally, R. Scopes, Protein Purification, Springer-Verlag, N.Y. (1982), Deutscher, Methods in Enzymology Vol. 182: Guide to Protein Purification, Academic Press, Inc. N.Y. (1990). The actual conditions used will depend, in part, on factors such as net charge, hydrophobicity, hydrophilicity, etc., and will be apparent to those having skill in the art. The functional properties may be evaluated using any suitable assays.

The functional properties of the fusion protein may be evaluated using any suitable assay such as antibody binding, induction of T cell proliferation, stimulation of cytokine production such as IL2, IL-4 and IFN-γ. For the practice of the present invention, it is preferred that each fusion protein is at least 80% purified from other proteins. It is more preferred that they are at least 90% purified. For in vivo administration, it is preferred that the proteins are greater than 95% purified.

The purified proteins may be further processed before use. For example, the proteins may digested with a specific enzyme to separate the Ra12 polypeptide from the heterologous polypeptide.

One of skill would recognize that modifications can be made to the recombinant nucleic acids and fusion polypeptides without diminishing their biological activity. Some modifications may be made to facilitate the cloning, expression, or incorporation of the tag molecule into a fusion polypeptide. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

The following Examples are offered by way of illustration and not by way of limitation.

### EXAMPLES

The following examples describe experiments that illustrate that Ra12 fusion constructs produced stable and high yield expression of fusion polypeptides. The following examples also illustrate that various Ra12 sequences can be used as a fusion partner.

### Example 1

### The Full Length Ra12 Sequence (SEQ ID NO:4) as a Fusion Partner

A. Construction of Expression Vectors

Coding sequences of M. tuberculosis antigens were modified by PCR in order to facilitate their fusion and subsequent expression of fusion protein. pET 17b vector (Novagen) was modified to include Ra12, a 14 kDa C-terminal fragment of the serine protease antigen MTB32A of M. tuberculosis. The 3′ stop codon of the Ra12 sequence was substituted with an in frame EcoRI site and the N-terminal end was engineered to code for six His-tag residues immediately following the initiator Met to facilitate a simple one step purification protocol of Ra12 recombinant proteins by affinity chromatography over Ni-NTA matrix.

Specifically, the C-terminal fragment of antigen MTB32A was amplified by standard PCR methods using the oligonucleotide primers 5′ CAA TTA CAT ATG CAT CAC CAT CAC CAT CAC ACG GCC GCG TCC GAT AAC TTC (SEQ ID NO:13) and the 3′ oligonucleotide sequence is 5′-CTA ATC GAA TTC GGC CGG GGG TCC CTC GGC CAA (SEQ ID NO:14). The 450 bp product was digested with NdeI and EcoRI and cloned into the pET 17b expression vector similarly digested with the same enzymes. Expression of the recombinant Ra12 protein was accomplished following transformation into the E. coli BL-21 (pLysE) host cells (Novagen) and induction with IPTG. Following lysis of the E. coli cells and centrifugation at 10K rpm, recombinant Ra12 was found in the soluble supernatant fraction. Protein from the soluble supernatant was purified by affinity chromatography over an Ni-NTA column which remained soluble following dialysis in 1×PBS. The amount of purified protein obtained was routinely in the 60 to 100 mg per liter range.

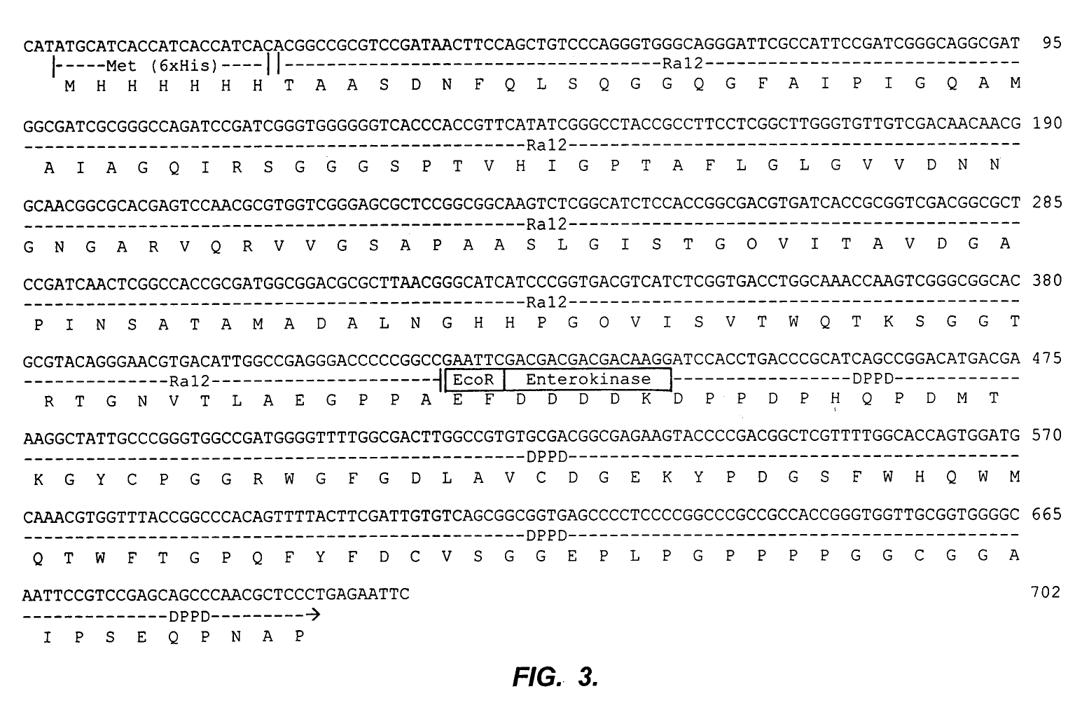

DPPD sequence was engineered for expression as a fusion protein with Ra12 by designing oligonucleotide primers to specifically amplify the mature secreted form. The 5′ oligonucleotide containing an enterokinase recognition site (DDDK) has the sequences 5′-CAA TTA GAA TTC GAC GAC GAC GAC AAG GAT CCA CCT GAC CCG CAT CAG-3′ (SEQ ID NO:15) and the 3′ oligonucleotide sequence is 5′ CAA TTA GAA TTC TCA GGG AGC GTT GGG CTG CTC (SEQ ID NO:16). The resulting PCR amplified product was digested with EcoRI and subcloned into the EcoRI site of the pET-Ra12 vector. Following transformation into the E. coli host strain (XL1-blue; Stratagene), clones containing the correct size insert were submitted for sequencing in order to identify those that were in frame with the Ra12 fusion. Subsequently, the DNA of interest (FIG. 3) was transformed into the BL-21 (pLysE) bacterial host and fusion protein expressed following induction of the culture with IPTG.

B. Expression and Purification of Fusion Proteins

The recombinant (His-tag) Ra12-DPPD fusion protein was purified from 500 ml of IPTG induced batch cultures from the soluble supernatant by affinity chromatography using the one step QIAexpress Ni-NTA Agarose matrix (QIAGEN, Chatsworth, Calif.) in the presence of 8M urea. Briefly, 20 ml of an overnight saturated culture of BL21 containing the pET construct was added into 500 ml of 2×YT media containing 50 ug/ml ampicillin and 34 ug/ml chloramphenicol, grown at 37° C. with shaking. The bacterial cultures were induced with 2 mM IPTG at an OD 560 of 0.3 and grown for an additional 3 h (OD 1.3 to 1.9). Cells were harvested from 500 ml batch cultures by centrifugation and resuspended in 20 ml of binding buffer (0.1 M sodium phosphate, pH 8.0; 10 mM Tris-HCl, pH 8.0) containing 2 mM PMSF and 20 ug/ml leupeptin. E. coli was lysed by adding 15 mg of lysozyme and rocking for 30 min at 4° C. following sonnication (4×30 sec). Lysed cells were spun at 12 k rpm for 30 min and urea was added directly to the supernatant at a final concentration of 8M.

The supernatant was batch bound to Ni-NTA agarose resin (5 ml resin per 500 ml inductions) by rocking at R/T for 1 h and the matrix passed over a column. The flow through was passed twice over the same column followed by three washes with 30 ml each of wash buffer (0.1 M sodium phosphate and 10 mM Tris-HCL, pH 6.3) also containing 8 M urea. Bound protein was eluted with 30 ml of 100 mM imidazole in wash buffer and 5 ml fractions collected. Fractions containing the recombinant antigen were pooled, dialyzed against 10 mM Tris-HCl (pH 8.0) bound one more time to the Ni-NTA matrix, eluted and dialyzed in 1×PBS (pH 7.4) or 10 mM Tris-HCL (pH 7.8). The yield of the purified recombinant fusion protein was in the 50 to 75 mg per liter of induced bacterial culture with greater than 95% purity representing a single band. Recombinant proteins were assayed for endotoxin contamination using the Limulus assay (Bio Whittaker) and were shown to contain <10 E.U./mg (<1 ng LPS/mg).

C. Generation of Antiserum

The purified fusion protein (100 ug) was mixed with 100 ug of muramyl dipeptide, brought up to 1 ml with 1×PBS and emulsified with 1 ml IFA (incomplete freunds; Life Technologies) adjuvant. The emulsion was injected at multiple sites s.c. into a female New Zealand rabbit (R&R Rabbitry, Stanwood, Wash.). The rabbit was given two subsequent boosters (100 ug antigen in IFA) 6 weeks apart and a final i.v. shot with 100 ug of the recombinant protein again given after 6 weeks. One week after the final boost, the rabbit was sacrificed and serum was collected and stored at −20° C.

D. Immunoblotting Analysis

M. tuberculosis (strain H37Rv) total lysate or PPD (2.5 μg each) and 25 ng of the purified recombinant Ra12-DPPD fusion protein were separated by electrophoresis on 16% SDS-PAGE gels and transferred to nitrocellulose using a semi-dry transfer apparatus (BioRad). Blots, in duplicate, were blocked for a minimum of 1 hr with PBS/0.1% Tween and probed with polyclonal sera from the same rabbit prior to immunization or post immunization with the purified recombinant fusion protein (diluted 1:500 in PBS/0.1% Tween 20). Reactivity was assessed as previously using [¹²⁵I]-protein A, followed by autoradiography.

E. Results

Several expression systems were initially evaluated for the expression of DPPD in E. coli. This included sub-cloning of DPPD coding sequence as non-fusion constructs in 1) pET 17b (Novagen) and pQ30 (Qiagen, Santa Clarita, Calif.) or 2) as fusion constructs using pET32A (Novagen, Madison, Wis.) or pGEX-2T (Pharmacia Biotech, Piscataway, N.J.). In all of these systems, very little if any DPPD was expressed and purified.

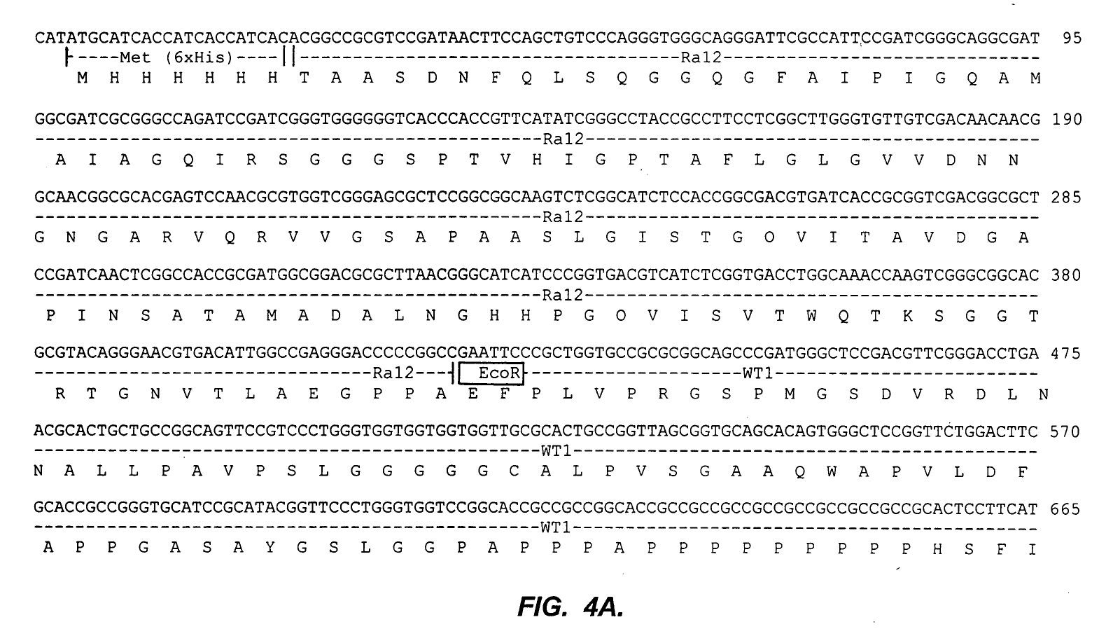

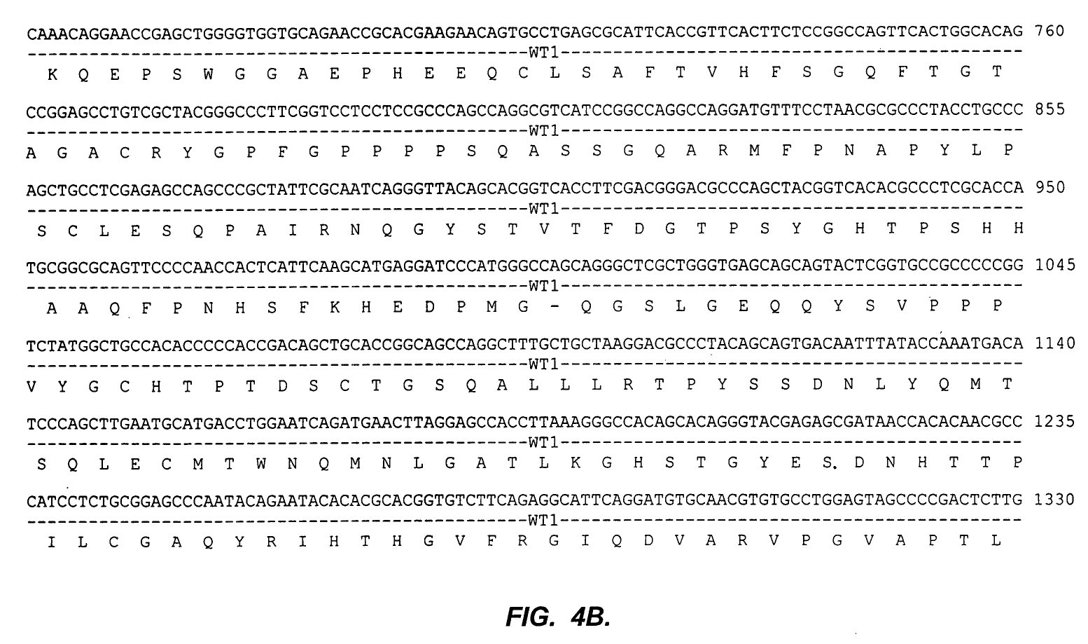

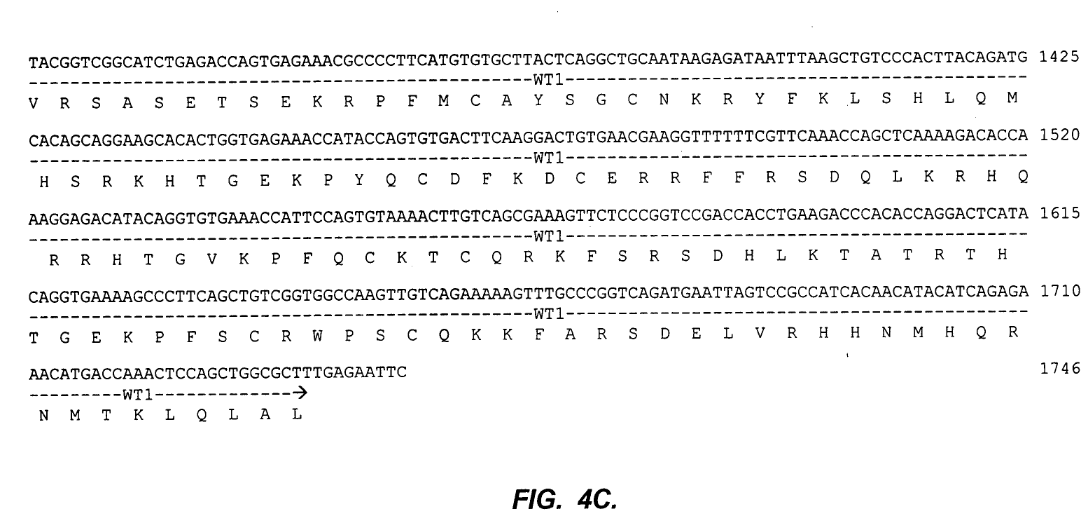

In contrast, when the DPPD coding sequence was inserted 3′ to the Ra12 sequence in an expression vector and transformed into E. coli, a large amount of Ra12-DPPD fusion protein was produced. The nucleotide sequence (SEQ ID NO:5) and amino acid sequence (SEQ ID NO:6) of Ra12-DPPD are disclosed in FIG. 3. The immunogenicity of DPPD was maintained as evidenced by the ability of antiserum to react with the purified protein in immunoblotting analysis. In addition, three other proteins of eukaryotic or prokaryotic origin (see FIGS. 4-6) were also successfully expressed by the Ra12 fusion constructs. Thus, the Ra12 coding sequence is useful as a fusion partner in an expression construct to facilitate the expression of a heterologous sequence.

### Example 2

### Short Ra12 Polypeptide (SEQ ID NO:17) as a Fusion Partner

In this example, a Ra12 polypeptide comprising amino acids 1-30 of SEQ ID NO:4 was used as a fusion partner to link with the full length human mammaglobin gene. This short form of Ra12 polypeptide has the amino acid sequence shown in SEQ ID NO:17, and is referred to herein as “Ra12(short)”.

As shown in FIG. 9, the 3′ end of the Ra12(short) sequence is fused to the full length human mammaglobin gene. Specifically, the human mammaglobin gene was amplified by standard PCR methods using the following oligonucleotide primers: the 5′ primer, Hind III site: 5′-gcgaagcttATGAAGTTGCTGATGGTCCTCATGC-3′ (SEQ ID NO:19); the 3′ primer, XhoI site: 5′-cggctcgagTTAAAATAAATCACAAAGACTGCTGTC-3′ (SEQ ID NO:20). The 5′ Hind III and 3′ Xho I sites were added to assist subcloning into a vector. The N-terminal end of the fusion construct was engineered to code for six His-tag residues immediately following the Met to facilitate purification protocols. The expression of the fusion construct was accomplished following transformation into E. coli using procedures similar to those described in Example 1. Compared to a construct without a Ra12(short) sequence, the fusion construct with a Ra12(short) sequence substantially increased the expression of the fusion Ra12(short)-mammaglobin protein.

### Example 3

### Longer Ra12 Polypeptide (SEQ ID NO:18) as a Fusion Partner

In this example, a Ra12 polypeptide comprising amino acids 1-128 of SEQ ID NO:4 was used as a fusion partner to link with the full length human mammaglobin gene. This long form of Ra12 polypeptide has the amino acid sequence shown in SEQ ID NO:18, and is referred to herein as “Ra12(long)”. Cloning and expression procedures similar those described in Example 2 were used. Compared to a construct without a Ra12(long) sequence the fusion construct with a Ra12(long) sequence substantially increased the expression of the fusion Ra12(long)-mammaglobin protein.

The present invention is not to be limited in scope by the exemplified embodiments which are intended as illustrations of aspects of the invention, and any clones, nucleotide or amino acid sequences which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims. It is also to be understood that all base pair sizes given for nucleotides are approximate and are used for purposes of description.

All publications cited herein are incorporated by reference in their entirety.

1-16. (canceled)

17. A fusion polypeptide comprising a Ra12 polypeptide and a heterologous polypeptide, wherein the Ra12 polypeptide is encoded by a Ra12 polynucleotide sequence that hybridizes to SEQ ID NO:3 under stringent hybridization conditions.

18. The fusion polypeptide according to claim 17, wherein the Ra12 polypeptide comprises at least about 10 amino acids.

19. The fusion polypeptide according to claim 17, wherein the Ra12 polypeptide comprises at least about 30 amino acids.

20. The fusion polypeptide according to claim 17, wherein the Ra12 polypeptide comprises at least about 100 amino acids.

21. The fusion polypeptide according to claim 17, wherein the Ra12 polypeptide has a sequence as shown in SEQ ID NO:4.

22. The fusion polypeptide according to claim 17, wherein the Ra12 polypeptide has a sequence as shown in SEQ ID NO:17.

23. The fusion polypeptide according to claim 17, wherein the Ra12 polypeptide has a sequence as shown in SEQ ID NO:18.

24. The fusion polypeptide of claim 17, the fusion polypeptide further comprising a linker peptide between the Ra12 polypeptide and the heterologous polypeptide.

25. The fusion polypeptide of claim 17, wherein the fusion polypeptide further comprises an affinity tag which is linked to the fusion polypeptide.

26. The fusion polypeptide of claim 17, wherein the heterologous polypeptide is a DPPD, a WT1, a mammaglobin, or a H9-32A.

27-31. (canceled)

32. The fusion polypeptide according to claim 17, wherein the Ra12 polypeptide has a sequence as shown in SEQ ID NO:23.
